(19) United States
(12) Patent Application Publication
(10) Pub. No.: US 2023/0039490 A1
(43) Pub. Date: Feb. 9, 2023

(54) LUNG BREATHING CHIP AND CELL STRETCHING CULTURE PLATFORM AND OPERATING METHOD THEREOF

(71) Applicants: NATIONAL TSING HUA UNIVERSITY, Hsinchu City (TW); TAIPEI MEDICAL UNIVERSITY, Taipei City (TW)

(72) Inventors: Cheng-Yu HUANG, Taichung City (TW); Chun-Hui SUNG, Zhudong Township (TW); Pin-Tzu LAI, Dacun Township (TW); Chia-Ming HSU, Mailiao Township (TW); Yi-Ying LIANG, Guangzhou (CN); Kang-Yun LEE, Taipei City (TW); Shu-Chuan HO, New Taipei City (TW); Weilun SUN, New Taipei City (TW); Cheng-Hsien LIU, Hsinchu (TW)

(73) Assignees: NATIONAL TSING HUA UNIVERSITY, Hsinchu City (TW); TAIPEI MEDICAL UNIVERSITY, Taipei City (TW)

(21) Appl. No.: 17/397,073

(22) Filed: Aug. 9, 2021

(30) Foreign Application Priority Data
Aug. 4, 2021 (TW) 110128774

(51) Int. Cl. C12M 3/00; C12M 3/06; C12M 1/36; C12M 1/00; C12N 5/00; G01N 33/50; C12N 5/071; C12M 1/42

(52) U.S. Cl. CPC C12M 21/08; C12M 23/16; C12M 23/46; C12M 29/10; C12M 35/04; C12M 41/48; C12N 5/0062; C12N 5/0697; G01N 33/5088; C12N 2513/00; C12N 2527/00

(57) ABSTRACT

A lung breathing chip and cell stretching culture platform and an operating method thereof are disclosed. The lung breathing chip and cell stretching culture platform controls the output of the motor by programming, stretches the micro-fluidic chip by the cam component, changes the size of the cam component and the frequency of the motor rotation to change the stretching frequency and the amount of stretching to simulate the breathing of the lungs in different states, uses liquid electrophoresis technology to arrange the cells in the biocompatible hydrogel and the hydrogel three-dimensionally to imitate the three-dimensional cell tissue, and injects drugs through the dynamic perfusion system to realize the drug testing platform that the cells of the chip bionic lung tissue are stretched.

### BACKGROUND OF THE INVENTION

### 1. Field of the Invention

The invention relates to cell culturing; in particular, to a lung breathing chip and cell stretching culture platform and operating method thereof.

### 2. Description of the Prior Art

For drug research and development and etiological research, compared with conventional animal experimental bionic chips, since human organ bionic chips can directly use human cells and can be customized, their development is of great significance.

For example, the lung is an important organ for the human body to breathe. However, in the development and design of the conventional lung bionic chip, it is still faced with the inability to set the chip stretching system in a cell incubator for operation to simulate lung breathing. This problem needs to be overcome urgently.

In general, microfluidic chips usually use an injection syringe to inject cells into the chip system through a plastic tube. However, the disadvantage of this method is that it is difficult to accurately control the flow rate and the number of injected cells and it is difficult to make the cells uniformly distributed in a designated area, it also needs to be overcome urgently.

In addition, conventionally, if the cells are to be arranged in a specific pattern, the chip structure is usually changed or the photocurable hydrogel is used in combination with a photomask. However, these methods have larger limitations in size, which leads to their poor practicality.

Furthermore, since cells usually produce some cytotoxins during the growth process, if the cells are cultured for a long time, the nutrients in the culture solution will be completely digested and the cytotoxins will accumulate, which will not only cause damage to the cells, but also this static culture environment is difficult to simulate the actual growth environment of the cells in the patient's body, and it needs to be overcome urgently.

### SUMMARY OF THE INVENTION

Therefore, the invention provides a lung breathing chip and cell stretching culture platform and operating method thereof to effectively solve the above-mentioned problems in the prior art.

An embodiment of the invention is a lung breathing chip and cell stretching culture platform. In this embodiment, the lung breathing chip and cell stretching culture platform includes: a microfluidic chip, on which a plurality of cells is accommodated; a motor, wherein an output of the motor is controlled by programming; and a cam element, configured to stretching the microfluidic chip. A stretching frequency and a stretching amount of the microfluidic chip are changed by changing a size of the cam element and a rotation frequency of the motor to simulate breathing of a lung in different states. The plurality of cells on the microfluidic chip is three-dimensionally arranged through liquid dielectrophoresis technology to imitate a three-dimensional cell tissue.

In one embodiment, the lung breathing chip and cell stretching culture platform further includes a dynamic perfusion system configured to perfuse drugs to perform drug testing on the three-dimensional cell tissue which is stretched.

In one embodiment, a microfluidic structure on the microfluidic chip combines photocuring of a three-dimensional biocompatible hydrogel and liquid dielectrophoresis technology to arrange the plurality of cells in three dimensions, and co-cultivating different cell tissues to completely simulate lung tissue environment in the patient, the three-dimensional biocompatible hydrogel system is used as a cell culture environment to simulate cell matrix and cytoskeleton in a human body, and the dynamic perfusion system is used to replace culture medium to simulate a state of blood flow in the human body.

In one embodiment, when the microfluidic chip is stretched, the holes in a porous film of the microfluidic chip are also deformed.

In one embodiment, the microfluidic chip includes a plurality of flow channels for simultaneously observing results of different lung simulation experiments.

Another embodiment of the invention is a method for operating a lung breathing chip and cell stretching culture platform. In this embodiment, the operating method includes the following steps: (a) controlling an output of a motor through programming; (b) using a cam element to stretch a microfluidic chip; (c) changing a size of the cam element and a rotation frequency of the motor to change a stretching frequency and a stretching amount of the microfluidic chip to simulate lung breathing in different states; and (d) using liquid dielectrophoresis technology to perform three-dimensional arrangement of a plurality of cells on the microfluidic chip to imitate a three-dimensional cell tissue.

In one embodiment, the method further includes the following steps: (e) perfusing drugs through a dynamic perfusion system to perform drug testing on the three-dimensional cell tissue which is stretched.

In one embodiment, the step (d) further includes: using the microfluidic structure on the microfluidic chip combined with photocuring of three-dimensional biocompatible hydrogel and liquid dielectrophoresis technology to three-dimensionally arrange the plurality of cells to co-culture different cell tissues to completely simulate lung tissue environment in the patient, where the three-dimensional biocompatible hydrogel system is used as a cell culturing environment to simulate cell matrix and cytoskeleton in a human body, and the dynamic perfusion system is used to replace culturing medium to simulate a state of blood flow in the human body.

In one embodiment, when the microfluidic chip is stretched, holes in a porous film of the microfluidic chip are also deformed.

In one embodiment, the microfluidic chip includes a plurality of flow channels for simultaneously observing results of different lung simulation experiments.

Compared with the prior art, the lung breathing chip and cell stretching culture platform and operating method thereof proposed in the invention can achieve the following advantages and effects:
(1) construction of lung tissue microenvironment: the invention uses microfluidic structure combined with three-dimensional biocompatible hydrogel and liquid dielectrophoresis technology to arrange cells, and co-culturing different cells to completely simulate the lung tissue environment in the patient.
(2) dynamic perfusion system: the invention uses biocompatible hydrogel as a cell culturing environment to simulate cell matrix and cytoskeleton in the human body, and uses the dynamic perfusion system to replace culturing medium to simulate the state of blood flow in the human body to make the growth environment of the cells in the chip closer to the environment in the human body, to make the cells grow effectively, to achieve the purpose of bionics, and to greatly extend the life of the cells to increase the observation and culturing time.
(3) cell stretching system: through the motor frequency control and cam size selection in the cell stretching device, the invention can respectively simulate different frequencies and different cell stretching amounts during human breathing. Different sizes of cam elements can be combined with servo motors to achieve different stretching amounts and stretching frequencies, and the holes on the stretched chip will also be deformed.
(4) dielectrophoresis chip: the dielectrophoresis chip used in the invention can control cells very well, by applying voltage to arrange the cells in the designated electrode area, and can achieve uniform distribution of the cells, and cooperate with photocuring hydrogel to fix the cell without a mask, the operation is simple, the experimental operation steps are saved, the cost and time are saved, and the utilization rate of the cells can be greatly increased, and the waste of the cells can be reduced.
(5) multi-channel design: the invention can realize simultaneous observations of the results of a variety of different lung tissue simulation experiments, such as “lung tissue affected by air pollution”, “lung tissue not affected by air pollution”, “Lung tissue is stretched” and “Lung tissue is not stretched” through the design of multiple channels (such as four-channel) to greatly shorten the simulation time.

The advantage and spirit of the invention may be understood by the following detailed descriptions together with the appended drawings.

### BRIEF DESCRIPTION OF THE APPENDED DRAWINGS

FIG. 1A and FIG. 1B show side views of the microfluidic chip before and after being stretched respectively.

FIG. 2A to FIG. 2D show a schematic diagram of the microfluidic channel design in the microfluidic chip, a schematic diagram of the microfluidic chip combined with a servo motor, a top view of the upper microfluidic channel, and a top view of the lower microfluidic channel respectively.

FIG. 3A to FIG. 3C show a side view, a top view and a perspective view of the porous film in the microfluidic chip respectively.

FIG. 4 shows a schematic diagram of a lung breathing chip and cell stretching culture platform.

FIG. 5 is a schematic diagram showing the movement of the linkage mechanism driven by the rotation of the cam.

FIG. 6 shows schematic diagrams before and after the holes of the porous film are deformed by different types of cams.

FIG. 7 shows a schematic diagram of the liquid dielectrophoresis electrode design.

FIG. 8A to FIG. 8D show schematic diagrams of a lung tissue bionic chip combined with air pollution suspended particles, a schematic diagram of a lung tissue bionic chip combined with a chip stretching system, a schematic diagram of an upper microchannel, and a schematic diagram of a lower microchannel.

FIG. 9 shows a flowchart of the method of operating the lung breathing chip and cell stretching culture platform.

### DETAILED DESCRIPTION OF THE INVENTION

Exemplary embodiments of the invention are referenced in detail now, and examples of the exemplary embodiments are illustrated in the drawings. Further, the same or similar reference numerals of the components/components in the drawings and the detailed description of the invention are used on behalf of the same or similar parts.

An embodiment of the invention is a lung breathing chip and cell stretching culture platform and its operating method. It has advantages of compactness and portability, simple operation, etc., which can effectively solve the conventional problem that the chip stretching system fails to be operated in the cell incubator.

In practical applications, in order to enable bionic chips such as lung breathing chips to provide more ideal performance, cell culturing in the bionic chips is also an urgent problem to be overcome. Therefore, the invention utilizes liquid dielectrophoresis technology to effectively overcome the conventional shortcomings of the inability to effectively arrange cells during cell co-cultivation, and uses the photocuring properties of hydrogel to solve the problem of cell positioning in the bionic chip and simplify operation steps of the conventional cell fixation method.

In addition, the invention also combines the biocompatible hydrogel with the cells through liquid dielectrophoresis technology to construct a three-dimensional cell mass, thereby different from the conventional two-dimensional cell culture method. Furthermore, the invention also uses porous membranes and dynamic perfusion technology for cell culture, so that the growth environment of cells in vitro can be closer to the actual situation in the human body, thereby enhancing the practicability and application value of the bionic chip of the invention.

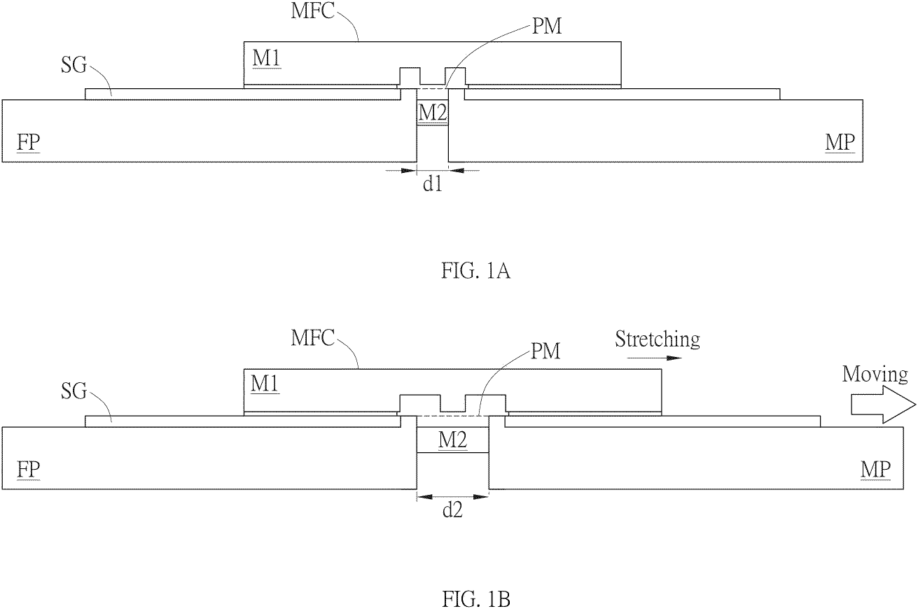

Please refer to FIG. 1A and FIG. 1B. FIG. 1A and FIG. 1B show a side view of the microfluidic chip before and after being stretched respectively. As shown in FIG. 1A, when the microfluidic chip MFC has not been stretched, the left and right sides of the upper microchannel M1 of the microfluidic chip MFC are respectively disposed above the two stretched glass slides SG and the middle part of the upper microchannel M1 of the microfluidic chip MFC is disposed above the porous film PM. The two stretched glass slides SG are respectively disposed above the fixed part FP and the movable part MP. The lower microchannel M2 is disposed between the fixed part FP and the movable part MP and is located under the porous film PM. When the microfluidic chip MFC has not been stretched, the distance between the fixed part FP and the movable part MP is d1; that is to say, the length of the lower microchannel M2 is d1.

As shown in FIG. 1B, when the right side of the microfluidic chip MFC is stretched, the movable part MP located under it moves to the right, while the fixed part FP located under the left side of the microfluidic chip MFC remains immobile. Therefore, the distance between the fixed part FP and the movable part MP will increase from d1 to d2; that is to say, the length of the lower microchannel M2 will also increase from d1 to d2. In addition, the porous film PM located under the middle part of the upper microchannel M1 will also be extended.

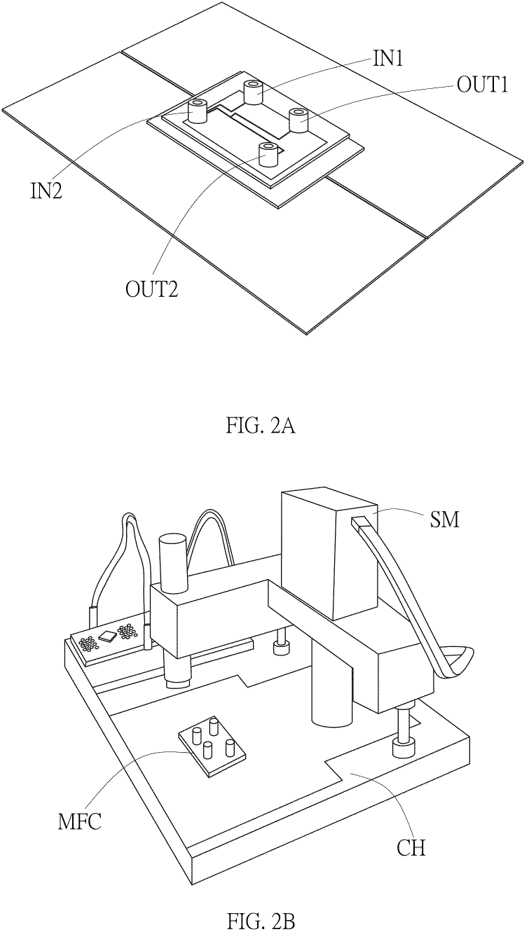

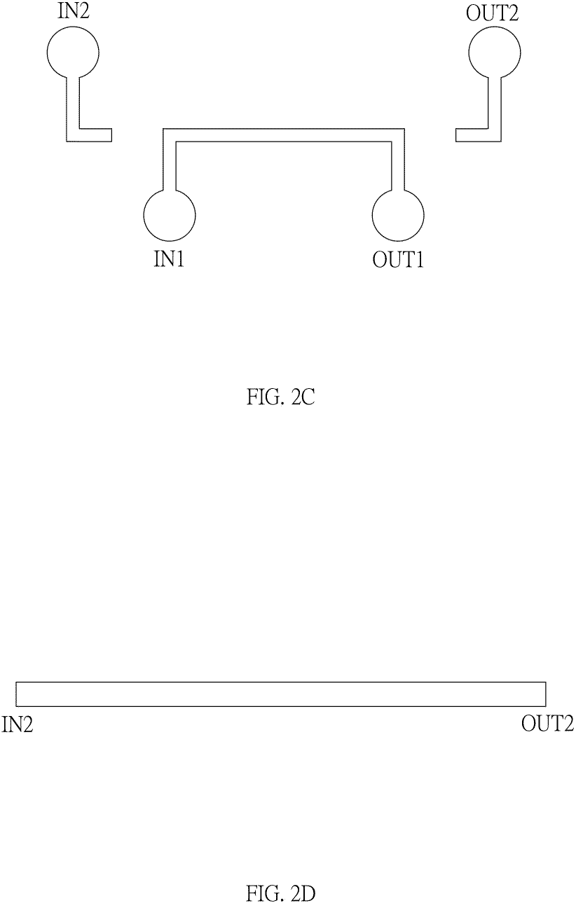

Please refer to FIG. 2A to FIG. 2D. FIG. 2A to FIG. 2D show a schematic diagram of the microfluidic channel design in the microfluidic chip, a schematic diagram of the microfluidic chip combined with a servo motor, a top view of the upper microfluidic channel, and a top view of the lower microfluidic channel respectively.

As shown in FIG. 2A, the microfluidic chip of the invention is designed with a double-layer microchannel (for example, the upper microchannel and the lower microchannel). FIG. 2B shows a schematic diagram of the microfluidic chip MFC and the servo motor SM integrated on the chip holder CH. The upper views of the upper microchannel and the lower microchannel are shown in FIG. 2C and FIG. 2D respectively. The upper microchannel is connected to the upper inlet IN1, the upper outlet OUT1, the lower inlet IN2 and the lower outlet OUT2 respectively, and the lower microchannel is connected to the lower inlet IN2 and lower exit OUT2 respectively.

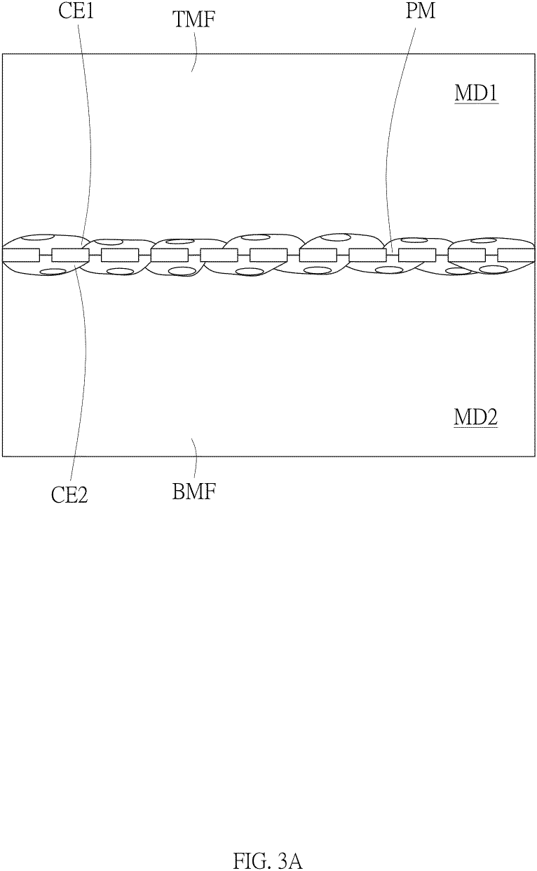

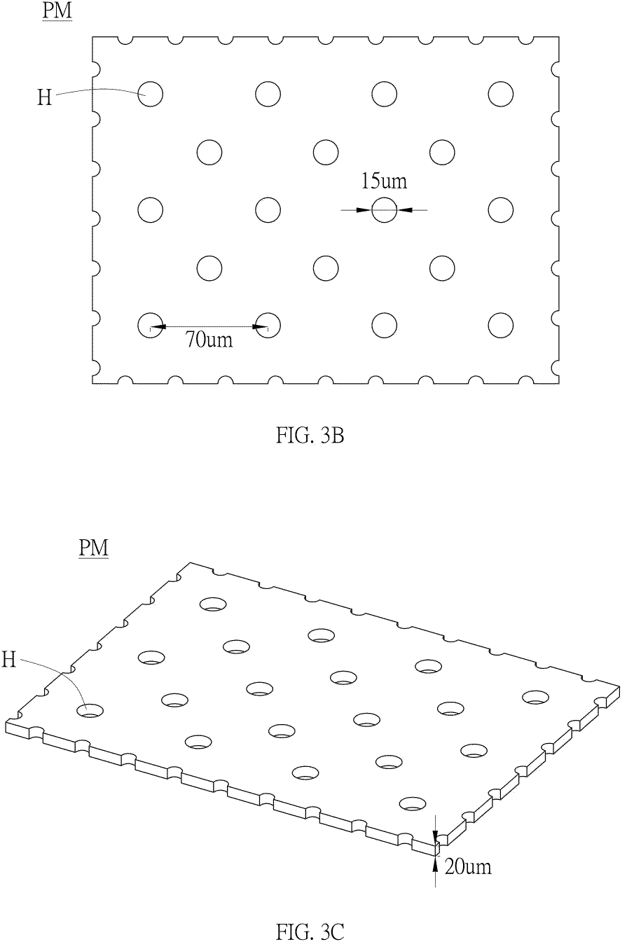

Please refer to FIG. 3A to FIG. 3C. FIG. 3A to FIG. 3C show a side view, a top view and a perspective view of the porous film in the microfluidic chip respectively.

As shown in FIG. 3A, the porous film PM is located between the upper microchannel TMF and the lower microchannel BMF. Assume that there are a first medium MD1 and a plurality of first cells CE1 in the upper microchannel TMF and a second medium MD2 and a plurality of second cells CE2 in the lower microchannel BMF. The plurality of first cells CE1 are attached to the top of the porous film PM and the plurality of second cells CE2 are attached to the bottom of the porous film PM.

In fact, the plurality of first cells CE1 and the plurality of second cells CE2 may be different types of cells, and the first medium MD1 and the second medium MD2 may be different types of cell-compatible media (for example, Hydrogel, but not limited to this).

As shown in FIG. 3B and FIG. 3C, the porous film PM includes a plurality of holes H. The plurality of holes H may have a diameter of 15 um and are arranged in a staggered manner. The distance between two adjacent holes H can be 70 um. The height of the porous film PM can be 20 um.

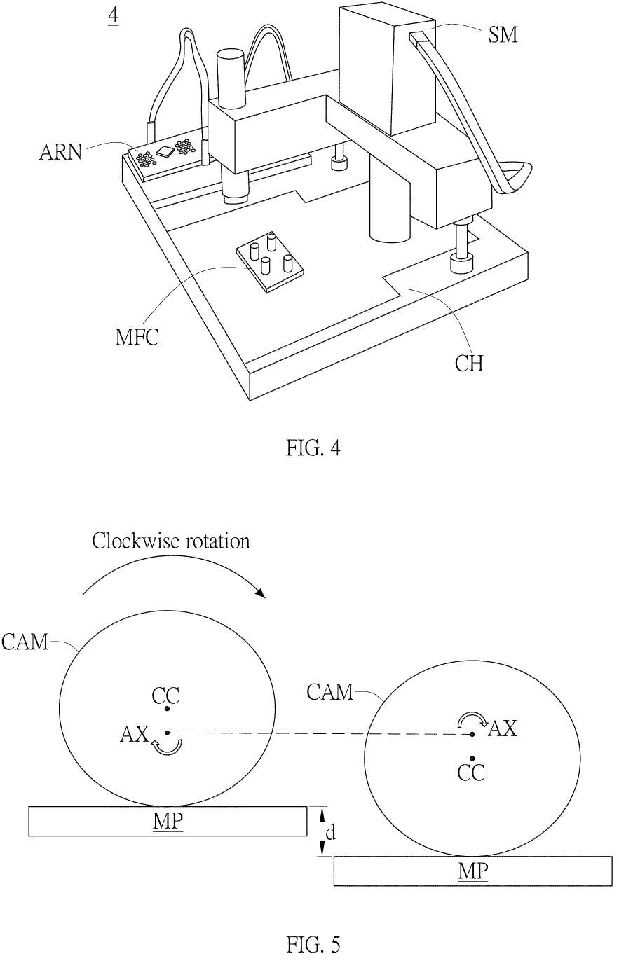

Please refer to FIG. 4, which shows a schematic diagram of the lung breathing chip and cell stretching culture platform. As shown in FIG. 4, the lung breathing chip and cell stretching culture platform 4 include a microfluidic chip MFC, a servo motor SM, a chip holder CH and a circuit board ARN. The chip holder CH is used to carry the microfluidic chip MFC. The circuit board ARN is used to electrically connect the microfluidic chip MFC and the servo motor SM. The servo motor SM is used to stretch the cells in the microchannel on the microfluidic chip MFC to simulate the condition of human breathing.

Please refer to FIG. 5, which illustrates a schematic diagram of the movement of the linkage mechanism driven by the rotation of the cam. As shown in FIG. 5, since the center CC of the cam CAM is different from the fixed axis AX, when the cam CAM rotates clockwise, the fixed axis AX remains stationary and the center CC of the cam CAM rotates accordingly, which will drive the linkage mechanism (the movable part MP) to move down a distance d, but not limited to this.

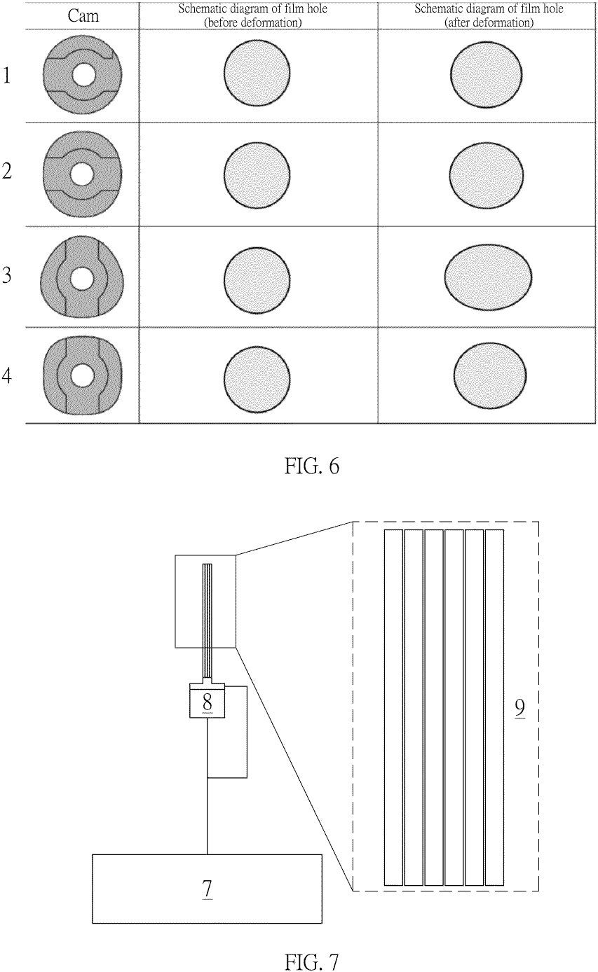

Please refer to FIG. 6. FIG. 6 shows a schematic diagram of different types of cams before and after the holes of the porous film are deformed. As shown in FIG. 6, the cam used in the invention can be (1) an eccentric cam, (2) an elliptical cam, (3) a triangular cam or (4) a rectangular cam. When it rotates, it can make different deformations occur in the holes H of the porous film PM in FIG. 3B, for example, as shown in the schematic diagram of the holes before and after the deformation shown in FIG. 6, but not limited to this.

Please refer to FIG. 7. FIG. 7 shows a schematic diagram of the design of the liquid dielectrophoresis electrode. As shown in FIG. 7, 7 is the voltage input point, 8 is the initial storage area of the cells, and 9 is the area where the cells are arranged and fixed.

It should be noted that conventional microfluidic chips usually use injection syringes to inject cells into the chip system through a plastic tube, but it often causes disadvantages such as difficult to control the flow rate, fail to accurately grasp the number of cells, and difficult to make the cells uniformly distributed in the designated area. In addition, if the cells are to be arranged in a specific pattern, the chip structure is usually changed or the photocuring hydrogel is used to bond the photomask, but both will have larger restrictions on size.

In contrast, the dielectrophoresis chip used in the invention can finely manipulate the cells, and evenly arrange the cells in the designated electrode area by applying a voltage, and is used with photocuring hydrogel, can be photocured without a mask. Because it is easy to operate and can greatly increase the utilization rate of cells, it can effectively save cost and time, and can also avoid cell waste.

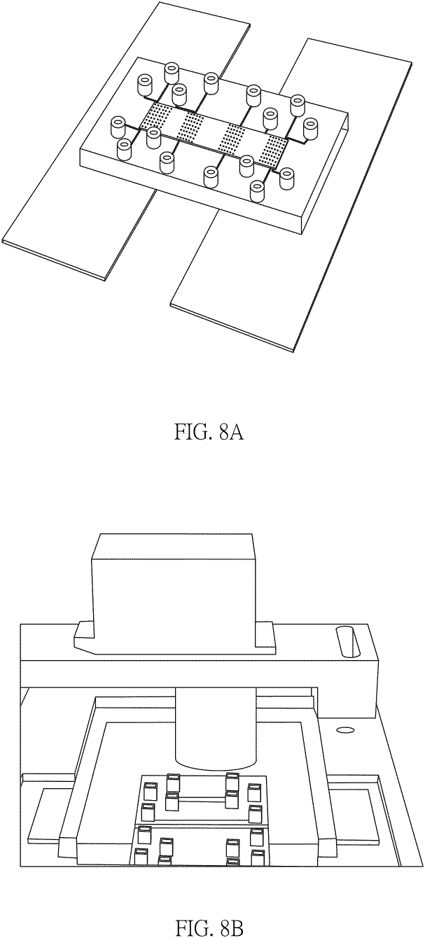

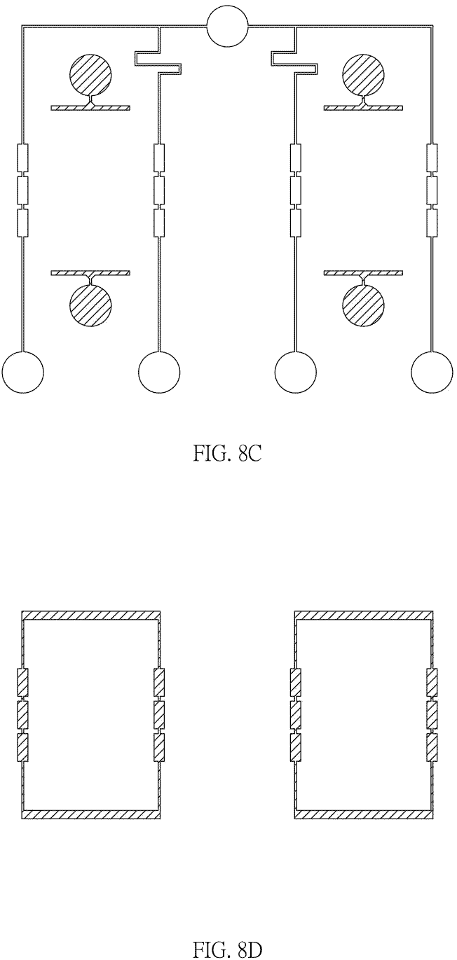

Please refer to FIG. 8A to FIG. 8D. FIG. 8A to FIG. 8D show a schematic diagram of a lung tissue bionic chip combined with air pollution suspended particles, a schematic diagram of a lung tissue bionic chip combined with a chip stretching system, a schematic diagram of an upper microchannel and a schematic diagram of a lower microchannel.

As shown in FIG. 8A and FIG. 8B, by introducing air pollution suspended particles, the lung tissue bionic chip can be simulated on the lung tissue bionic chip to simulate the influence of air pollution on the lung tissue under the stretching action of the chip stretching system. FIG. 8C and FIG. 8D show schematic diagrams of the upper microchannel and the lower microchannel respectively. For example, the four micro-channels included in the upper microchannel can be used to simultaneously observe the results of four different lung tissue simulation experiments, such as “the lung tissue is affected by air pollution”, “the lung tissue is not affected by air pollution”, “the lung tissue is stretched” and “the lung tissue is not stretched” to save time and improve efficiency.

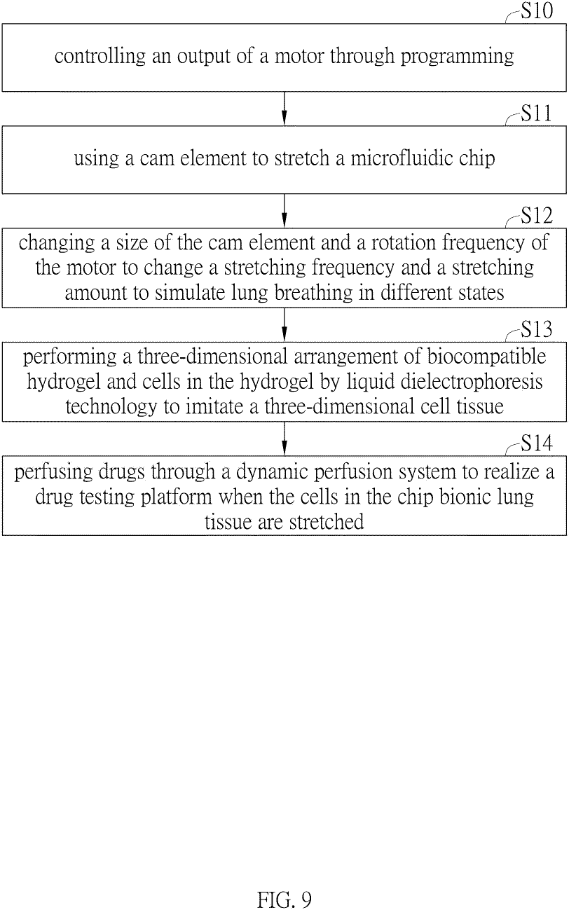

Please refer to FIG. 9. FIG. 9 shows a flow chart of the method of operating the lung breathing chip and cell stretching culture platform. As shown in FIG. 9, the method can include the following steps S10~S14, but not limited to this:
Step S10: controlling an output of a motor through programming;
Step S11: using a cam element to stretch a microfluidic chip;
Step S12: changing a size of the cam element and a rotation frequency of the motor to change a stretching frequency and a stretching amount to simulate lung breathing in different states;
Step S13: performing a three-dimensional arrangement of biocompatible hydrogel and cells in the hydrogel by liquid dielectrophoresis technology to imitate a three-dimensional cell tissue; and
Step S14: perfusing drugs through a dynamic perfusion system to realize a drug testing platform when the cells in the chip bionic lung tissue are stretched.

Compared with the prior art, the lung breathing chip and cell stretching culture platform and operating method thereof proposed in the invention can achieve the following advantages and effects:
(1) construction of lung tissue microenvironment: the invention uses microfluidic structure combined with three-dimensional biocompatible hydrogel and liquid dielectrophoresis technology to arrange cells, and co-culturing different cells to completely simulate the lung tissue environment in the patient.
(2) dynamic perfusion system: the invention uses biocompatible hydrogel as a cell culturing environment to simulate cell matrix and cytoskeleton in the human body, and uses the dynamic perfusion system to replace culturing medium to simulate the state of blood flow in the human body to make the growth environment of the cells in the chip closer to the environment in the human body, to make the cells grow effectively, to achieve the purpose of bionics, and to greatly extend the life of the cells to increase the observation and culturing time.
(3) cell stretching system: through the motor frequency control and cam size selection in the cell stretching device, the invention can respectively simulate different frequencies and different cell stretching amounts during human breathing. Different sizes of cam elements can be combined with servo motors to achieve different stretching amounts and stretching frequencies, and the holes on the stretched chip will also be deformed.
(4) dielectrophoresis chip: the dielectrophoresis chip used in the invention can control cells very well, by applying voltage to arrange the cells in the designated electrode area, and can achieve uniform distribution of the cells, and cooperate with photocuring hydrogel to fix the cell without a mask, the operation is simple, the experimental operation steps are saved, the cost and time are saved, and the utilization rate of the cells can be greatly increased, and the waste of the cells can be reduced.
(5) multi-channel design: the invention can realize simultaneous observations of the results of a variety of different lung tissue simulation experiments, such as “lung tissue affected by air pollution”, “lung tissue not affected by air pollution”, “Lung tissue is stretched” and “Lung tissue is not stretched” through the design of multiple channels (such as four-channel) to greatly shorten the simulation time.

What is claimed is:

1. A lung breathing chip and cell stretching culture platform, comprising:
a microfluidic chip, on which a plurality of cells is accommodated;
a motor, wherein an output of the motor is controlled by programming; and
a cam element, configured to stretching the microfluidic chip;
wherein, a stretching frequency and a stretching amount of the microfluidic chip are changed by changing a size of the cam element and a rotation frequency of the motor to simulate breathing of a lung in different states; the plurality of cells on the microfluidic chip is three-dimensionally arranged through liquid dielectrophoresis technology to imitate a three-dimensional cell tissue.

2. The lung breathing chip and cell stretching culture platform of claim 1, further comprising:
a dynamic perfusion system, configured to perfuse drugs to perform drug testing on the three-dimensional cell tissue which is stretched.

3. The lung breathing chip and cell stretching culture platform of claim 1, wherein a microfluidic structure on the microfluidic chip combines photocuring of a three-dimensional biocompatible hydrogel and liquid dielectrophoresis technology to arrange the plurality of cells in three dimensions, co-cultivating different cell tissues to completely simulate lung tissue environment in the patient, the three-dimensional biocompatible hydrogel is used as a cell culture environment to simulate cell matrix and cytoskeleton in a human body, and the dynamic perfusion system is used to replace culture medium to simulate a state of blood flow in the human body.

4. The lung breathing chip and cell stretching culture platform of claim 1, wherein when the microfluidic chip is stretched, the holes in a porous film of the microfluidic chip are also deformed.

5. The lung breathing chip and cell stretching culture platform of claim 1, wherein the microfluidic chip comprises a plurality of flow channels for simultaneously observing results of different lung simulation experiments.

6. A method of operating a lung breathing chip and cell stretching culture platform, comprising steps of:
(a) controlling an output of a motor through programming;
(b) using a cam element to stretch a microfluidic chip;
(c) changing a size of the cam element and a rotation frequency of the motor to change a stretching frequency and a stretching amount of the microfluidic chip to simulate lung breathing in different states; and
(d) using liquid dielectrophoresis technology to perform three-dimensional arrangement of a plurality of cells on the microfluidic chip to imitate a three-dimensional cell tissue.

7. The method of claim 6, further comprising:
(e) perfusing drugs through a dynamic perfusion system to perform drug testing on the three-dimensional cell tissue which is stretched.

8. The method of claim 6, wherein the step (d) further comprising:
using the microfluidic structure on the microfluidic chip combined with photocuring of three-dimensional biocompatible hydrogel and liquid dielectrophoresis technology to three-dimensionally arrange the plurality of cells to co-culture different cell tissues to completely simulate lung tissue environment in the patient, where the three-dimensional biocompatible hydrogel system is used as a cell culturing environment to simulate cell matrix and cytoskeleton in a human body, and the dynamic perfusion system is used to replace culturing medium to simulate a state of blood flow in the human body.

9. The method of claim 6, wherein when the microfluidic chip is stretched, holes in a porous film of the microfluidic chip are also deformed.

10. The method of claim 6, wherein the microfluidic chip comprises a plurality of flow channels for simultaneously observing results of different lung simulation experiments.
